(19) United States
(12) Patent Application Publication
(10) Pub. No.: US 2006/0241765 A1
(43) Pub. Date: Oct. 26, 2006

(54) Load bearing intervertebral disk

(76) Inventors: Peter James Burn, CHRISTCHURCH (NZ); Lain Alexander McMillan, Christchurch (NZ)

Correspondence Address: JACOBSON HOLMAN PLLC, 400 SEVENTH STREET N.W., SUITE 600, WASHINGTON, DC, 20004, US

(21) Appl. No.: 10/552,017

(22) PCT Filed: Apr. 5, 2004
(86) PCT No.: PCT/NZ04/00069

§ 371 Date: Jan. 3, 2006

(30) Foreign Application Priority Data
Apr. 3, 2003 (NZ) 525179
May 20, 2003 (NZ) 526019

(51) Int. Cl. A61F 2/44

(52) U.S. Cl. 623/17.12; 623/17.16; 623/23.41

(57) ABSTRACT

An implant useful as a prosthetic replacement of an intervertebral disk which consists of a housing providing at least a part simulation of a disk yet containing a force transferring media. The housing is in the form of a bellows or equivalent structure preferably restricted as to maximum separation between those surfaces of the housing to be in contact with the proximate vertebral bodies thereby to limit angular distortion under the effect of the proximate vertebral bodies.

### TECHNICAL FIELD

The present invention relates to load bearing structures.

There have been a number of devices proposed for use as disk replacements. There are none that meet all of the preferences above.

The present invention in one aspect relates to intervertebral disk replacement (including replacement for similar low-displacement joints). Since, therefore, there is contemplation of body joints (including vertebral facet joints or vertebral joints to the pelvis, etc.) the terms thereafter “intervertebral”, “proximal vertebral body”, etc. should take as an option corresponding meanings.

A collapsed disk can cause continuous pain, partial paralysis and limited mobility. If the situation is serious enough the current medical solution is for the patient to have a “spinal fusion” operation in which the collapsed disk is removed and, by one of several similar methods, the adjoining vertebrae are induced to grow together and in time become fused. This operation is successful in reducing the paralysis and pain but limits the flexibility of the spine and puts more load on to adjacent disks. As a result there is increased potential for the neighbouring disks to degenerate as well.

### BACKGROUND

The Sought-After Characteristics in a Disk Replacement:

The ideal replacement would be a component:

1. That will bond to the vertebrae.
2. That will have the flexibility in all respects similar to that of a healthy disk. This involves vertical and angular flexibility.
3. It should have stability similar to a healthy disk (for example it should not allow the adjacent vertebra to slide fore and aft or laterally in relation to one another).
4. It should spread the load into the vertebrae much in the manner of healthy disk.
   Other preferences are that:
5. It should be no more difficult to insert that the hardware at present used in the operation.
6. It should if possible use the same procedures as presently used.
7. Once in place it should have good survivability.
8. If anything should go wrong, it should not fail catastrophically.
9. It should be able to be replaced by hardware used in current methods.
10. It should not have sliding surfaces that produce wear debris.
11. It should be eminently biocompatible

Whilst there have been a number of devices mooted to be used as disk replacements, none has all of the preferences above.

An object of this invention in some of its embodiments is to meet at least several (if not all) of these criteria and thereby provide for the surgeon and patient a realistic alternative to spinal fusion as the method of choice for dealing with a collapsed intervertebral disk or to at least provide an alternative. Similarly with other joint application.

Another object of this invention in some of its embodiments is to meet all these criteria and thereby provide for the surgeon and patient a realistic alternative to spinal fusion as the method of choice for dealing with degenerate intervertebral disk.

The invention in one aspect in several forms is shown (cut in half) in FIGS. 13 through 20.

The invention in this form comprises a tough sealed outer casing in the form of a cushion or bellows arrangement filled with a bio compatible fluid medium (eg; liquid and/or otherwise) possibly incorporating a gas (eg; air) cavity or gas cavities. It may have other applications as discussed hereafter.

### BRIEF DESCRIPTION OF THE INVENTION

In one aspect the present invention consists in a load transferal device, said device being defined at least in part by

a space confinement housing which provides spaced exterior surface adapted to bear against surfaces of spaced members which are to have a capability over at least some distance and some angular disposition of moving relatively towards each other and/or angling relative to each other, and

at least one force transferring media within said housing.

Preferably said device is an implant useful for cushioning directly or indirectly bone members.

In another aspect the present invention consists in an implant useful as a prosthetic replacement of an intervertebral disk, said implant being defined at least in part by

a space confinement housing which provides, as an at least in part simulation of such a disk, top and bottom surfaces adapted (directly or indirectly) to bear at least in part respectively on the upper and lower vertebral bodies between which it might be interposed as an implant, and

at least one force transferring media within said housing,

wherein said housing under the influence of the confined media has a capability of allowing said top and bottom surfaces to be angularly disposed relative to each other in a number of different conditions (simulating those of an intervertebral disk) as a result of an ability of the housing under diverse loadings (such as those of real or simulated angular dispositions of proximate vertebral bodies between which the implant might be inserted) to compact in part and substantially correspondingly expand in part.

Preferably said implant has as a motion limiting feature (one or more) to restrict the maximum separation of said top and bottom surfaces.

Preferably said housing is in the form of a bellows or some equivalent (whether or unitary or fabricated form). Additional implant forms could include circular, spiral, horseshoe or banana profiles.

Preferably said housing is substantially of or is an adaption of a form substantially as depicted in any one or more of the accompanying drawings.

Preferably said media is at least in part liquid and/or at least in part gaseous and/or a resilient (at least in part) solid(s) material(s).

In another aspect the invention consists in the use of an implant as previously defined as a replacement for an intervertebral disk.

In yet another aspect the invention consists in a method of replacing an intervertebral disk which comprises or includes at least

(if necessary) opening the annulus,

(if necessary) removing the damaged or defective intervertebral disk, or residue thereof, and

interposing an implant as previously defined between the related proximal vertebral bodies,

(and if possible and/or necessary and/or desirable) restoring to its functional positioning the annulus),

(and, if the implant is not of a kind motion limited internally and/or externally of the housing to itself, at any appropriate stage, motion limiting the implant to one or other, or both, of said related proximal vertebral bodies).

In a further aspect the present invention consists in, interposed between, or for imposition between, vertebral bodies in a spinal structure, an implant as a prosthetic replacement of an intervertebral disk,

wherein said implant confines a force transferring media inside a housing capable of being compressed and/or angularly distorted by the effect of proximate vertebral bodies, whereby any such angular distortion, in part, compresses (and displaces some of the media) and, in part, expands (under the action at least in part of displaced media),

and wherein the media assisted expansion is limited by at least one of:
(i) motion limiters external of yet attached to the housing,
(ii) motion limiters internally of yet attached to the housing, and/or
(iii) motion limiters between each adjacent vertebral body and distal parts of the housing or motion limiters not attached to the housing.

### BRIEF DESCRIPTION OF THE DRAWINGS

Preferred forms of the present invention will now be described with reference to the accompanying drawings in which

FIG. 1 (two views, FIGS. 1A and 1B) shows simplified front and isometric pictorial views of normal spine anatomy and physiology,

FIG. 2 shows a simplified isometric view of position of a bellows type implant in accordance with the present invention relative to spine anatomy and physiology,

FIG. 3 (shows artists renderings of the lumbar spine anatomy and physiology (Source: Frank H. Netter, M.D.; Atlas of Human Anatomy; Plate 144), FIG. 3A showing the second lumbar vertebra: superior view, FIG. 3B shows the intervertebral disk, FIG. 3C shows the third and fourth lumbar vertebrae: posterior view and FIG. 3D shows the lumbar vertebrae, assembled: left lateral view),

FIG. 4A shows in cross section a bellows type housing in accordance with the present invention in a relaxed mode with two motion limiting devices on opposed sides each being in a relaxed state, and FIG. 4B shows the same view as in FIG. 4A but with an angular distortion as a result of applied force in the arrowed direction which has the effect of further relaxing the motion limiting device on the side to which the greater force is applied until such time as the other diametrically opposed motion limiting device pulls taut and those the forces Fₜ apply to limit further expansion, such limitation of further expansion ensuring there is sufficient force transferring media interposed on the more compressed side to prevent further and excessive contraction,

FIG. 5A (FIGS. 5A and 5B) show front and isometric pictorial views of a spine at rest with surrounding anatomy and physiology and a bellows implant (the bellows being partly obscured in FIG. 5B and an annulus comprised during insertion of the implant being shown with no thickness for clarity in the drawing),

FIG. 6 (FIGS. 6A and 6B) shows front and isometric pictorial views of a deflected spine with surrounding anatomy and physiology and bellows implant, these figures being similar to those of FIG. 5 but showing the nature of the performance,

FIG. 7 (FIGS. 7A and 7B) shows front and isometric pictorial views of a spine at rest with an externally tethered bellows implant (note the annulus has been omitted for clarity),

FIG. 8 (FIGS. 8A and 8B) shows front and isometric pictorial views of a deflected spine with externally tethered bellows implant (again the annulus being omitted for clarity),

FIG. 9 shows an isometric pictorial view of an internally tethered bellows type implant,

FIG. 10 shows an isometric pictorial view of an externally tethered bellows type implant,

FIG. 11 (FIGS. 11A and 11B) shows front and isometric pictorial views of a spine at rest with the vertebral bodies tethered (note the annulus has been omitted for clarity),

FIG. 12 (FIGS. 12A and 12B) shows front and isometric pictorial views of a deflected spine with the vertebral bodies tethered (note the annulus has been omitted for clarity),

FIG. 13 is a simple housing form shown in section that could be used as a compressible/expandable housing,

FIG. 14 shows an alternative form to that of FIG. 13 for such a housing again shown in section, this time as a bellows type housing having a number of concertinaing type convolutions,

FIG. 15 shows a more preferred form to that of FIG. 14 showing welded seams or (if integrally formed) sharper transitions in the concertina regions,

FIG. 16 is a variation of the housing of FIG. 13 showing vertebral body engagement projections,

FIG. 17 shows a variation of the embodiment of FIG. 13 having a plug capable of being fitted after filling with the force transferring media,

FIG. 18 is a further variation of the arrangement of FIG. 13 showing how the force transferring media can be more than a mere bio-compatible liquid in that it can include compressible spheres or other shapes therein, such as a gas filled bubble form,

FIG. 19 shows a further variation of FIG. 13 where a compressible foam or like at least in part solid(s) material(s) media is provided as the force transferring media,

FIG. 20 shows how, if desired, some more resistant to compression forms can be provided internally as motion limiting device(s), such provisions being instead of and/or in addition to any other motion limiting in accordance with the present invention,

FIG. 21 is a plan view of bean shaped forms, and

FIG. 22 shows a device of the present invention being interposed as cushioning below a tibial tray.

### DETAILED DESCRIPTION OF THE INVENTION

This invention in one form is show (cut in half and without tethers) in FIG. 13 and described in detail below.

The housing in this form comprises a tough sealed outer casing in the form of a cushion or bellows arrangement filled with a biocompatible fluid medium possibly incorporating a gas cavity or cavities.

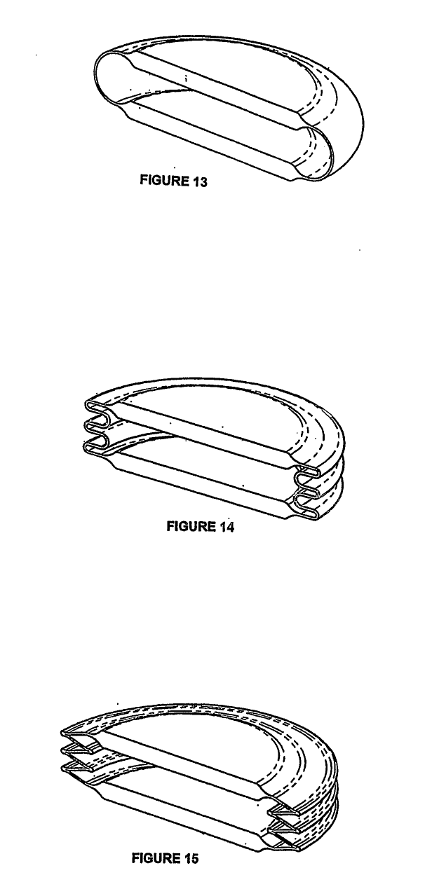

The housing of casing can be made of a suitable biocompatible material such as titanium or stainless steel. The outer edge of this structure could be thin and flexible, and may involve one or more convolutions (as for example in FIG. 14) depending on the requirements of space and flexibility. This design is not unlike flexible metal bellows found in steam piping. The bellows may be formed bellows as shown in FIG. 14 or edge-welded bellows, as shown in FIG. 15.

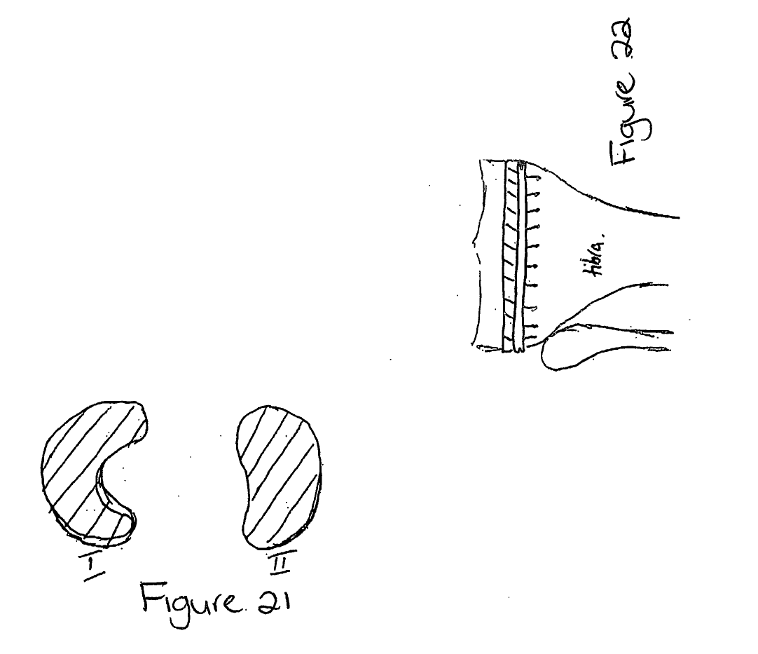

All the figures show the implant being circular, because this was the easiest configuration to draw however ideally it would be “bean” shaped. The plan view “bean” shape (as in FIG. 21) allows insertion from a posterior or more lateral approach—one being inserted from each side, or two from the same.

The vertebral bodies have increasing compressive strength closer to the periphery. Hence the geometry of the implant needs to mimic this profile to place the loads as close as possible this higher strength material thus ensuring the vertebral body endplates are not damaged, by the applied loads.

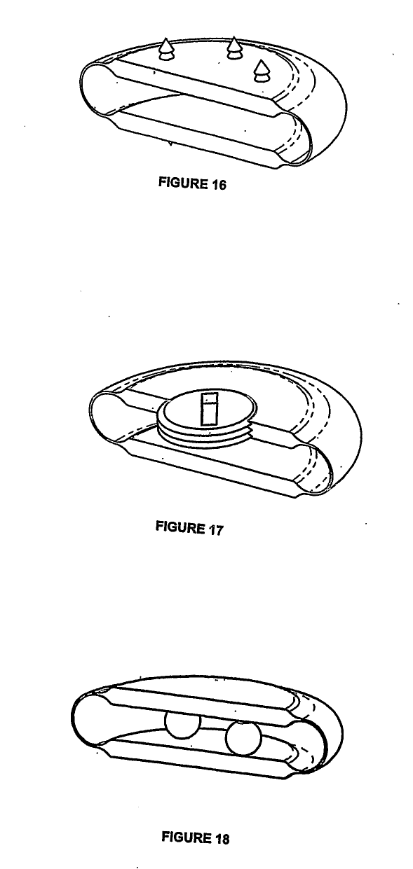

The ends of the structure could be thicker and might be coated (on the surfaces next to the bond) with a suitable bioactive agent or an appropriately roughened surface, the purpose of which is to encourage osteo-integration. They could contain barbed spikes (FIG. 16), deflectable spikes or the like, to help locate and fix them into the vertebral bodies during insertion.

The bellows wall may be coated

i) Internal coating to stop interacting of housing material and fill medium.

ii) Internal coating to help retain fill medium (fluid in this case) if a crack develops in the housing.

iii) Anodised outer surface to improve wear properties of implant should surrounding anatomy rub against implant (example similar treatment is applied to pacemakers).

The connection between the flexible sides and the thicker ends is suitably designed to avoid stress concentrations.

The ends may contain an opening filled for example with a flush screwed-in cap (FIG. 17) allowing insertion after manufacture of the internal medium

The housing confined force transferring media may take a number of forms. In its simplest for it could be gas (eg; air, nitrogen, CO₂, or the like), but this will not allow the structure to carry much load without collapse, and, should there be a crack in the outer casing, it would leak gas into the patient's body.

Alternatively it could be a simple liquid such as sterile saline. This would avoid the problem of gas leakage on rupture. However it would allow collapse of the casing structure if leakage occurred.

Having the casing completely filled with a fluid like saline would perhaps not provide sufficient compressibility, especially if the casing was metal. This however can be improved by providing a sealed plastic gas bubble or bubbles of know volume, floating within the fluid (FIG. 18). These would act as springs to any shock pressure loadings applied to the structure, and would provide some axial flexibility to the structure and prevent end plate overload and fracture.

Another alternative to the internal medium could be a flexible plastic material such as a suitable polyurethane or silicone polymer or the like. This would have the advantages that should cracking of the outer casing occur, the internal medium would not leak out but would retain its function for a considerable period before plastic creep and fretting cause problems. Compressibility of such a plastic filling could be ensured by embedding a gas cavity within it. This gas cavity could be purposefully chosen to be the right size and shape to give the chosen axial compressibility and rocking stiffness to the overall structure.

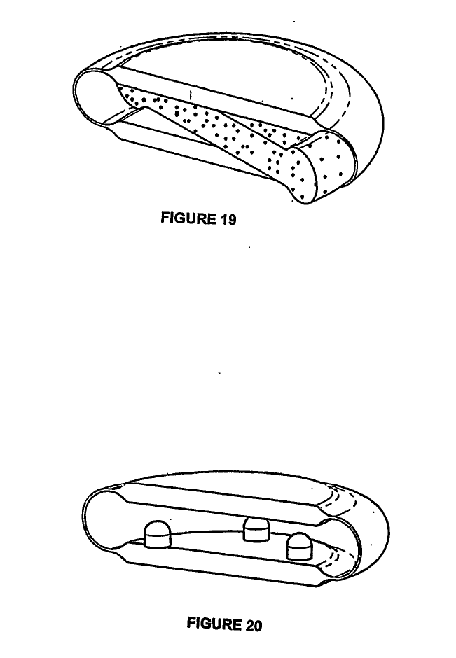

Another alternative as the internal medium could be a closed-cell foam of a chosen density (FIG. 19). Again this could have the advantages that it could be chosen to have the right resilience. Should cracking of the outer casing occur, this would not leak out but would retain its function, like the more solid plastic media.

The various internal media could have varying stiffness properties and/or the housing geometry could have varying sectional properties at different locations within the disk to suit the needs of rocking and vertical stiffness, and the compression of the bellows.

The structure could also include rigid internal buffer stops to prevent any overloading of the structure (FIG. 20).

A particular advantage of the structures of FIGS. 13 to 20 is that they emulate the behaviour of a live disk in the respect that:
a) it can carry large vertical loads by virtue of the fact that it has an internal fluid under pressure (albeit a somewhat plasticised fluid),
b) it has a small amount of axial compressibility,
c) it is relatively flexible to rocking motions,
d) it is very stiff to torsional motions, and
e) it is very stiff to shear between the top and bottom surfaces.

As a result of the semi fluid interior and the internal gas cavity(s), the cushion can be “tuned” to the required axial and rocking flexibilities by varying the stiffness of the internal material, and varying the size, shape and location of the gas cavity(s). As disk spaces are not always parallelograms in shape, the flexibility allows congruency.

A particular advantage of the structure is that it is completely enclosed within a casing such as titanium or a suitable alloy of titanium, which has long been used as an implant material.

Another particular advantage is that there are no external sliding surfaces producing friction and wear debris, known particularly with plastics, to cause toxic responses within the body.

Another advantage is that there are not titanium (or other metal) surfaces bearing on one another. As such it avoids the potential for wear, galling and wear debris with the titanium.

Another advantage over prior art is that this structure is relatively rigid against shear movements between the top and bottom ends. Unlike some earlier products, this design is able to maintain spinal stability with this feature.

Another particular advantage is that this cushion can be inserted in the same space and with the same procedures and operations as used currently for spinal fusion operations. In fact this design allows simpler insertion as it does not require to be filled with the bone particles required for spinal fusion.

A further advantages are that, should the unit need to be removed, a spinal fusion procedure can be implemented in its place—this design has not excluded a subsequent spinal fusion operation.

Another feature is that the large loads are carried by pressure in the internal fluid and this allows that walls of the cushion to the thin. This means that significant deflections can take place without raising the wall stress beyond critical values. This means the device can be designed to have a very long life in terms of metal fatigue.

Another is that since where internal material is biocompatible and solidified it means that if the outer titanium shell should crack, for example by fatigue or trauma, no material comes out, and there is no toxic reaction as a result. Further, since the gas cavity is well embedded within the plastic, no gas can escape into the body either. The wall thickness can be varied so critical area adjacent to the disk have greater protection in the event of a wall failure directing any extruded material away from this zone.

A related feature is that should failure occur, the consequences are not catastrophic. In fact that unit should still function with cracks through the thin wall. What is likely to happen is that the cracks would eventually develop wear debris that may be rejected by the body. The overall result is that should failure occur there is a long time available before the implant has to be replace, if at all.

While the designs described and drawn are intended primarily for the replacement of intervertebral disks they could also be used (with a suitable size alteration) in other body joints such as the vertebral facet joints. Importantly joints with complex movements (such as the wrist and subtarsal joints) could use this implant device with slight shape variations. It may also be incorporated under knee replacement implants or incorporated into the tibial tray design.

FIG. 22 shows a device in accordance with the present invention on a tibia below a tibial tray (eg; of CoCr metal) which itself is preferably below, for example, a polyethylene bearing.

Persons skilled in the art will appreciate other joint applications.

In a non medical application, i.e. industry it could be used for suspension systems, variable if the “bubble” volume were able to be adjusted during use.

The motion restricting systems described in this document are important to the success of the intervertebral implant for several reasons where there is no inherent mechanism within the bellows or other housing of the implant to prevent excessive deflection occurring. Over deflection could result in several issues, including:
a) Bellows implant being damaged causing immediate loss, or partial loss of function.
b) If the level of over deflection is minor, (i.e. there is no loss of function) but repeatedly occurs the implant life could be significantly reduced.
c) Bone/implant interface union could be damaged.
d) Surrounding anatomy and physiology could be damaged.

For these reasons motion-restricting systems have been developed to complement the bellows implant and prevent over deflection.

In the healthy lumbar spine there are several mechanisms that restrict the degree of deflection an intervertebral disc can travel through. These mechanisms include:

a) The surrounding ligaments and muscles groups

b) The facet joints

c) And the annulus

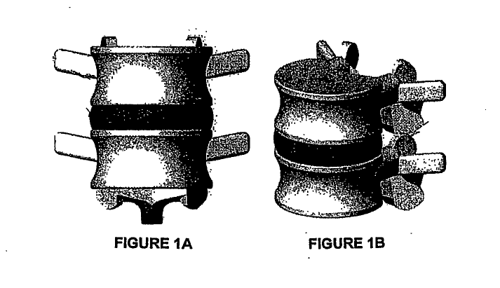

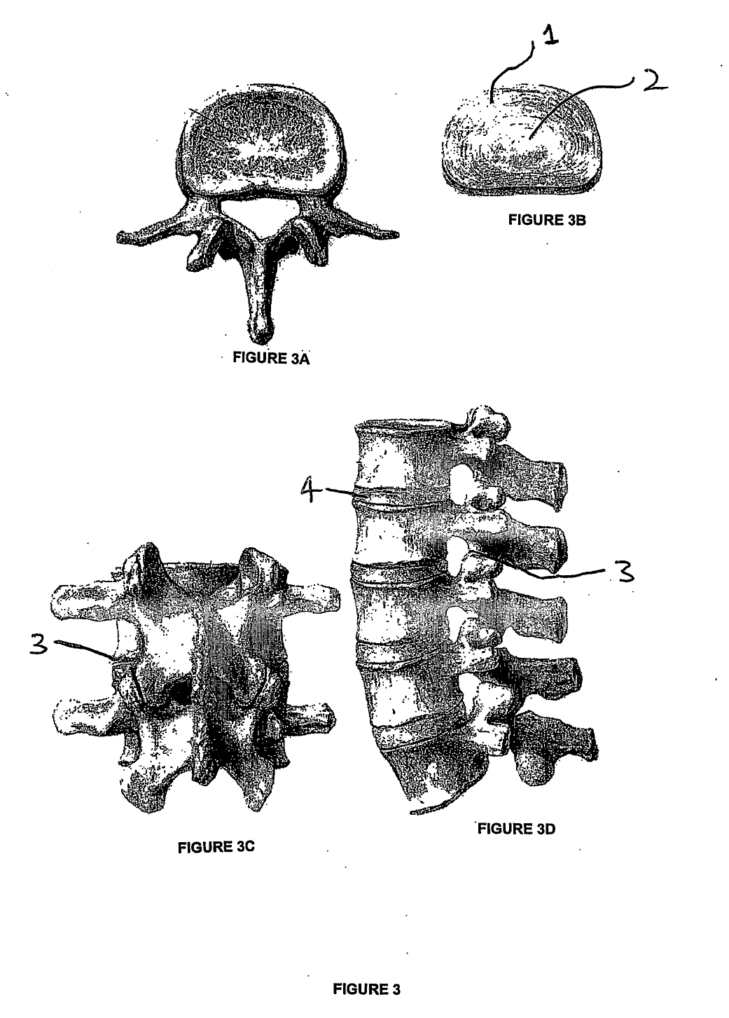

Shown in FIG. 1 is a simplified pictorial view of the lumbar spine. The intervertebral disc 4 consists of two “components” the annulus 1 and the nucleus pulposus 2 as shown in FIG. 3B.

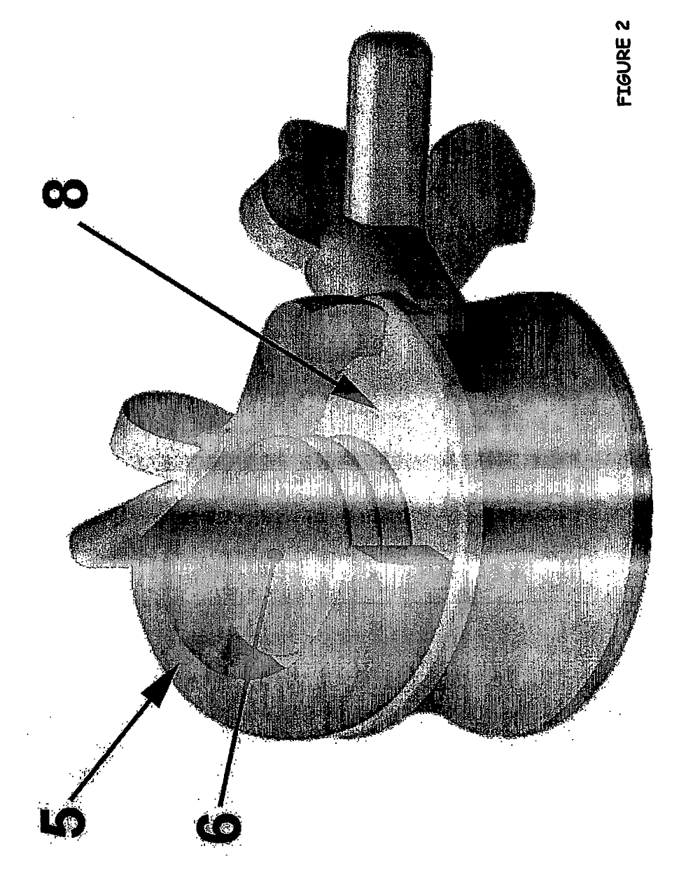

The bellows implant (as one example of a housing type) will normally be used in situations where the annulus is damaged and often the facet joints 3 may be damaged as well. During surgery approximately 30% of the annulus will be reflected to allow insertion of the bellows implant. The remaining nucleus will then be removed and the bellows implant 6 will sit in the nucleus cavity 8 surrounded by the remaining annulus 5, as shown in FIG. 2.

Therefore with two of the natural deflection restriction mechanisms potentially compromised, deflection of the bellows implant 6 must be able to controlled by adding other mechanisms to the implant.

To date, three different motion restriction mechanisms have been considered. These have included:

a) Bump Stops

b) Bellows Geometry

c) Tethers

Bump stops limit the maximum distance a mechanism can travel through. The bump stops may be configured as shown in FIG. 20, they may in either or both internally or externally of the bellows cavity.

The bump stops may be singular, plural, or continuous.

This method consists simply of two surfaces that come into contact when the maximum travel is reached. This method has the advantage of being very simple.

If the implant introduces a shearing motion when deflected, the bump stop surfaces will produce wear debris as they make contact. As the bellows implant will not introduce any shear motion, such wear debris would not be produced.

If the bump stops were situated within the housing cavity wear debris generation issues would be further minimised; as any such debris would be contained within the housing and would not able to make contact with the surrounding anatomy and physiology.

Such bump stops could also be designed to incorporated cushioning, reducing any impact loads as the bump stops come into contact.

Bump stops have been used in several intervertebral disc implants including Kostuik et al (U.S. Pat. No. 4,759,769).

The maximum deflection of the implant could also be controlled by the bellows geometry. Factors such as the bellows thickness, convolution pitch and convolution height all affect the level of deflection which results from an applied force. Therefore the bellows could be engineered to meet a specified maximum deflection.

Welded edge bellows as shown in FIG. 15 also offer the advantage of being able to deflect without incurring damage until the convolutions contact, or become “bound”, once bound no further deflection can occur.

This method would require a specific bellows for each patient based on his or her age, weight, muscle strength etc. Also the geometry of the bellows affects the peak stress the bellows experience and this stress in turn affects the life span of the bellows implant.

Tether systems are our favoured method for restricting the deflection of the implant.

Preferably there is a plurality of tethers.

Preferably said tethers are external of said housing.

Alternatively said tethers are internal of said housing.

In other forms the tethering can be distinct from the implant ie can be from one proximal vertebral bodies to the other.

In yet a different embodiment, (less preferred) there is a plurality of tethers with one or more attached to the housing towards one said surface for subsequent attachment to a vertebral body distal to said one said surface, and with one or more attached to the housing towards the other said surface for subsequent attachment to a vertebral body proximal to said one said surface.

Preferably said tethers are flexible ties that span between its existing or intended attachments so as not to provide any substantial fetter on compaction.

Preferably said tethers are flexible so as to not provide any substantial fetter on compaction.

Tether systems have been used in some conceptual intervertebral disc implant designs including Xavier et al (U.S. Pat. No. 6,063,121). It should also be noted that Kostuik et al (U.S. Pat. No. 4,759,769) also used tethers to prevent excessive extension of their implant.

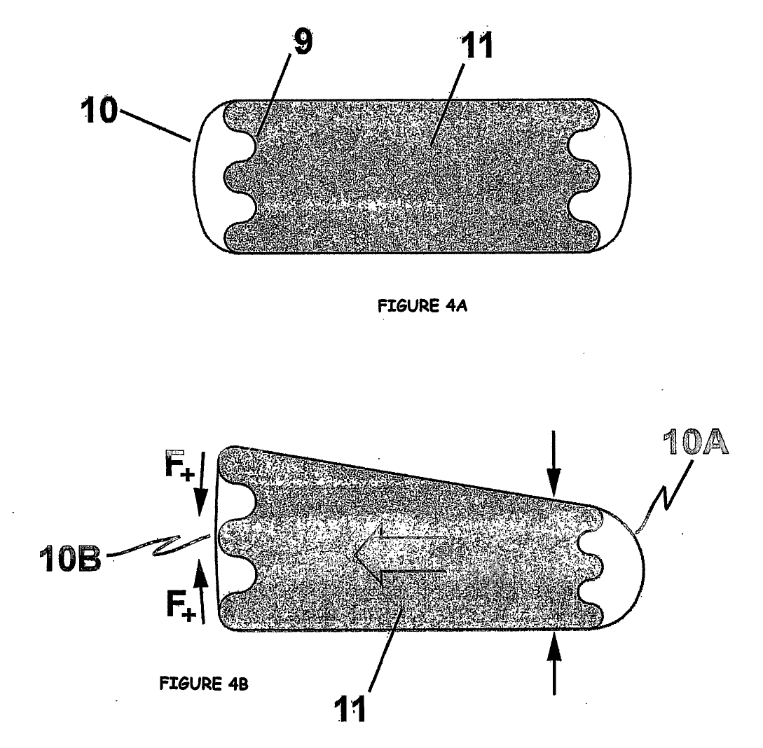

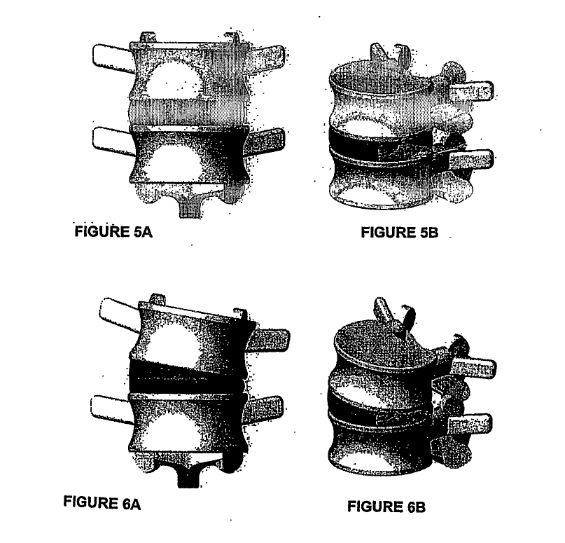

Tether systems work by taking advantage of the incompressible fluid within the bellows or other implant. See FIG. 4A. As the implant deforms one side of the disc implant is compressed as in FIG. 4B and at the least in part media inside the implant (eg; perhaps an incompressible confined liquid) is forced to the other side of the housing. This movement of the fluid 11 places the other side of the implant in tension. Around the periphery of the implant housing 9, or close to it, are fixed length tethers 10. As the disc deforms the tether 10A on the compressed side of the disc becomes relaxed and the tether 10B on the other side of the disk are placed in tension. When the tether 10B in tension becomes taut it stops any further skewing motion of the housing. Hence by controlling the length of all tethers 10 the maximum deflection on the opposing side can be fixed.

Tethers could be a continuous ring around the entire circumference (periphery) of the implant housing (i.e. not just discrete tethers) and hence the terms “at least one tether”, “one or more tethers”, etc encompasses such variations.

Several tether systems have been developed as an example which use this principle.

Use of Surrounding Anatomy Tether

This method of restricting motion requires no additions to the bellows implant and relies on the surrounding anatomy and physiology to limit deflections. In a healthy lumbar spine the annulus, facet joints, surrounding ligaments and muscles naturally act to restrict motion; hence this method is a continuation of this philosophy.

During insertion of the implant approximately 30% the annulus would have to be reflected however where possible the remainder of the annulus would be preserved.

The remaining annulus, facet joints etc would then be relied on to continue protecting the implant and anatomy and physiology from over deflection using the tether system as detailed in respect of FIGS. 4A and 4B.

This tethering system has the advantages of not adding complexity to the bellows implant design and using the patient's anatomy and physiology to the implants advantage. The potential problems with this method are that it may place unnecessary stress on the annulus, which will have already been weakened during implant insertion.

Externally Tethered Bellows

The bellows implant could be altered to include bands or a continuous ring of material around the outside of the bellows. These bands or ring would consist of a flexible, non-stretching material anchored to the implant. The length of the bands could be set at the time of manufacture to provide the required levels of deflection.

As for the previous method of tethering the bellows, “the surrounding anatomy and physiology tether”, the majority of the annulus would be left intact. In the views shown below the remaining annulus has been omitted for clarity.

Advantages of this system include, no reliance on the annulus to restrict movement, easy access to tether and the possibility of using the ring of material as a secondary fluid containment system, should the bellows fail. Disadvantages of this system include the need for the tether material to be biocompatible.

As the tether is not anchored to the bone if the patient continues to bend after the tether has become taut the vertebral bodies may separate from the implant damaging the implant/bone union. This separation of the implant and vertebral bodies is unlikely to occur though as the remaining annulus and ligaments will continue to limit deflection to safe levels as detailed previously.

Internally Tethered Bellows

The bellows implant design could also be altered to include bands or a continuous ring of material on the inside of the bellows. These bands or ring would consist of a flexible, non-stretching material anchored to the inside of the implant.

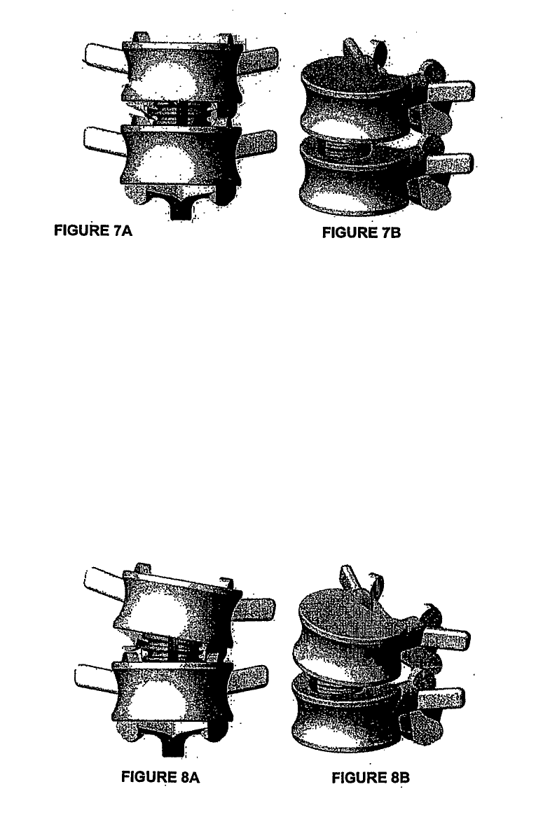

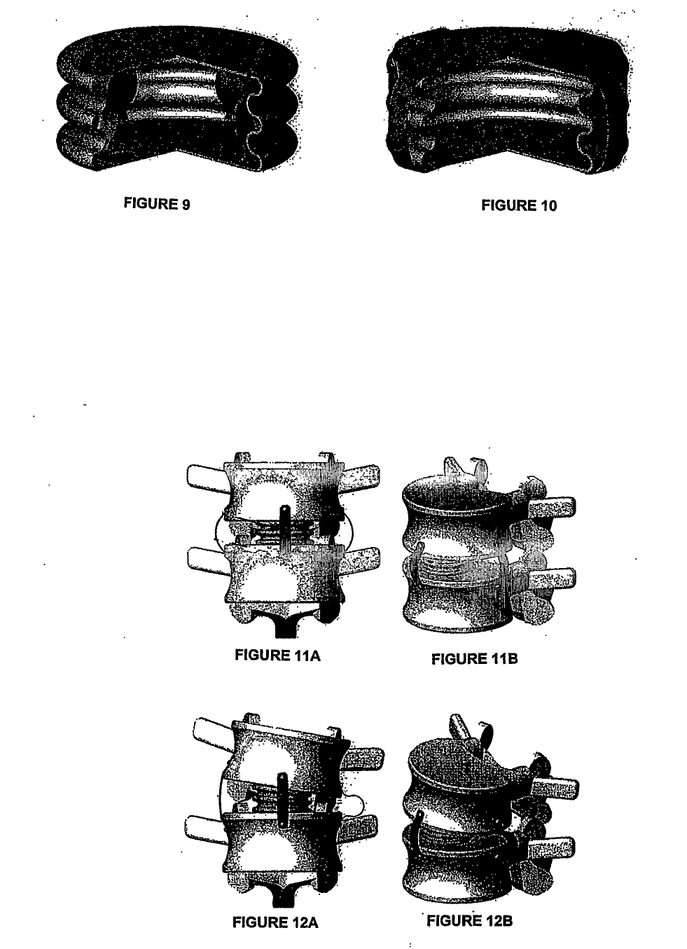

The internally and externally tethered bellows are inserted in an identical fashion. Hence the at-rest and deflected views of the internally tether bellows are very similar to the external tethered bellows shown in FIGS. 7 & 8.

Partial cross-sections of the internally and externally tethered bellows are shown below, as can be seen from these figures the implants are identical apart from the placement of tether.

Advantages of internal tethers include, the tether material need not be biocompatible and the possibility of using the ring of material as a system to contain the majority of the fluid should the bellows fail.

The major disadvantage of this tether system is increased difficulty accessing the tether material making construction potentially more difficult.

Once again as the tether is not anchored to the bone if the patient continues to bend after the tether has become taut the vertebral bodies may separate from the implant damaging the implant/bone union. This separation of the implant and vertebral bodies is unlike to occur though as the remaining annulus and ligaments will continue to limit motion to safe levels.

Independent Tether

This method of tethering the implant in effect recreates the annulus by fixing the tether rigidly to the vertebral bodies or other parts of the patients anatomy and physiology. This tether system would therefore prevent the patient from over deflecting hence removing the risk of the vertebral bodies separating from the implant and hence damaging the implant/bone union.

This method of tethering the implant would however dramatically increase the complexity of inserting the implant. The surgeon would be required to staple the tethers onto the vertebral bodies in the correct places and with the correct length of tether in each position. Due to lack of access to the lumbar spine anatomy during insertion of the implant, independent tethers could only be used to reinforce the reflected annulus. Independent tethers could also be used in conjuection with internally or externally tethered bellows, with the same restriction above.

It should be noted that the positions of the tethers on the periphery of the vertebral bodies in FIG. 11 & FIG. 12 are purely schematic and the tethers could be placed in other positions.

Suitable biocompatible tethering or other materials include any of those disclosed in the aforementioned patents. Attachment thereof to a housing can be by adhesion (eg; with a biocompatible adhesive) or otherwise. Attachment thereof to bone can be by adhesion, or screws, etc. See again aformentioned patents.

1. A load transferal device, said device being defined at least in part by a space confinement housing which provides spaced exterior surface adapted to bear against surfaces of spaced members which are to have a capability over at least some distance and some angular disposition of moving relatively towards each other and/or angling relative to each other, and at least one force transferring media within said housing.

2. The device of claim 1 which is an implant useful for cushioning directly or indirectly bone members.

3. An implant useful as a prosthetic replacement of an intervertebral disk, said implant being defined at least in part by
a space confinement housing which provides, as an at least in part simulation of such a disk, top and bottom surfaces adapted (directly or indirectly) to bear at least in part respectively on the upper and lower vertebral bodies between which it might be interposed as an implant, and
at least one force transferring media within said housing,
wherein said housing under the influence of the confined media has a capability of allowing said top and bottom surfaces to be angularly disposed relative to each other in a number of different conditions (simulating those of an intervertebral disk) as a result of an ability of the housing under diverse loadings (such as those of real or simulated angular dispositions of proximate vertebral bodies between which the implant might be inserted) to compact in part and substantially correspondingly expand in part.

4. The implant of claim 3 which has one or more motion limiting feature to restrict the maximum separation of said top and bottom surfaces.

5. The implant of claim 4 wherein said implant confines a force transferring media inside a housing capable of being compressed and/or angularly distorted by the effect of proximate vertebral bodies, whereby any such angular distortion, in part, compresses (and displaces some of the media) and, in part, expands under the action at least in part of displaced media),
and wherein the media assisted expansion is limited by at least one of:
(i) motion limiters external of yet attached to the housing, and/or
(ii) motion limiters internally of yet attached to the housing.

6. The device of claim 3, wherein said housing is in the form of a bellows or some equivalent (whether or unitary or fabricated form).

7. The device of claim 6 wherein said housing is substantially of or is an adaption of a form substantially as depicted in any one or more of the accompanying drawings.

8. The device of claim 4 said media is at least in part liquid and/or at least in part gaseous and/or a resilient (at least in part) solid(s) material(s).

9. The use of an implant defined in claim 4 as a replacement for an intervertebral disk.

10. A method of replacing an intervertebral disk which comprises or includes at least
(if necessary) opening the annulus,
(if necessary) removing the damaged or defective intervertebral disk, or residue thereof, and
interposing an implant as defined in any one of claims 4 to 8 between the related proximal vertebral bodies,
(and if possible and/or necessary and/or desirable) restoring to its functional positioning the annulus),
(and, if the implant is not of a kind motion limited internally and/or externally of the housing to itself, at any appropriate stage, motion limiting the implant to one or other, or both, of said related proximal vertebral bodies).

11. Interposed between, or for imposition between, vertebral bodies in a spinal structure, an implant as a prosthetic replacement of an intervertebral disk,
wherein said implant confines a force transferring media inside a housing capable of being compressed and/or angularly distorted by the effect of proximate vertebral bodies, whereby any such angular distortion, in part, compresses (and displaces some of the media) and, in part, expands (under the action at least in part of displaced media),
and wherein the media assisted expansion is limited by at least one of:
(i) motion limiters external of yet attached to the housing,
(ii) motion limiters internally of yet attached to the housing, and/or
(iii) motion limiters between each adjacent vertebral body and distal parts of the housing or motion limiters not attached to the housing.
